(19) United States
(12) Patent Application Publication
(10) Pub. No.: US 2024/0084399 A1
(43) Pub. Date: Mar. 14, 2024

(54) COMPOSITIONS AND METHODS FOR TREATING HEMATOLOGICAL CANCERS TARGETING THE SIRPALPHA - CD47 INTERACTION

(71) Applicants: UNIVERSITY HEALTH NETWORK, Toronto (CA); THE HOSPITAL FOR SICK CHILDREN, Toronto (CA)

(72) Inventors: Jean C.Y. WANG, Toronto (CA); John DICK, Toronto (CA); Jayne DANSKA, Toronto (CA); Liqing JIN, Toronto (CA); Alexandre THEOCHARIDES, Toronto (CA); Sujeetha RAJAKUMAR, Toronto (CA)

(73) Assignees: UNIVERSITY HEALTH NETWORK, Toronto, ON (CA); THE HOSPITAL FOR SICK CHILDREN, Toronto, ON (US)

(21) Appl. No.: 18/518,353

(22) Filed: Nov. 22, 2023

Related U.S. Application Data
(62) Division of application No. 16/118,038, filed on Aug. 30, 2018.
Application No. 16/118,038 is a continuation of application No. 13/320,629, filed on Apr. 2, 2012, now Pat. No. 10,907,209.
(60) Provisional application No. 61/178,553, filed on May 15, 2009.

(51) Int. Cl. C12Q 1/6886; A61K 38/16

(52) U.S. Cl. CPC C12Q 1/6886; A61K 38/16; C12Q 2600/156

(57) ABSTRACT

The invention relates to modulating the SIRPα-CD47 interaction in order to treat hematological cancer and compounds therefor. In some embodiments, there is provided methods and uses of SIRPα polypeptides, fragments and fusion proteins for treating hematological cancer, preferably human acute myeloid leukemia.

### RELATED APPLICATIONS

This application is a divisional of U.S. patent application Ser. No. 16/118,038, filed Aug. 30, 2018, which is a continuation of U.S. patent application Ser. No. 13/320,629, filed Apr. 2, 2012, now U.S. Pat. No. 10,907,209, which is a national phase application under 35 U.S.C. § 371 of International Application No. PCT/CA2010/000743, filed May 14, 2010, which claims the benefit of priority from U.S. Provisional Application No. 61/178,553, filed May 15, 2009, each of which applications are incorporated by reference herein in their entirety.

### SEQUENCE LISTING

The instant application contains a Sequence Listing which has been submitted in XML format and is hereby incorporated by reference in its entirety. Said XML copy, created on Nov. 22, 2023, is named “NRORP0003USC1D1-SeqListing.xml” and is 15,164 bytes in size.

### FIELD OF THE INVENTION

The invention relates to targeting the SIRPα-CD47 interaction in order to treat hematological cancer, particularly human acute myeloid leukemia (AML), and compounds therefor.

### BACKGROUND OF THE INVENTION

Graft failure in the transplantation of hematopoietic stem cells occurs despite donor-host genetic identity of human leukocyte antigens, suggesting that additional factors modulate engraftment. With the non-obese diabetic (NOD)-severe combined immunodeficiency (SCID) xenotransplantation model, it was recently found that the NOD background allows better hematopoietic engraftment than other strains with equivalent immunodeficiency-related mutations (Takenaka, K. et al. Polymorphism in Sirpa modulates engraftment of human hematopoietic stem cells. Nat. Immunol. 8, 1313-1323 (2007)). Polymorphisms in the Sirpa allele were identified and shown to be responsible for the differences in engraftment between the mouse strains analyzed. While the NOD background conferred the best support for human engraftment, mice with other polymorphisms of Sirpa could not be engrafted (i.e. NOD.NOR-Idd13.SCID). In mouse and human, Sirpa encodes for the SIRPα protein which interacts with its ligand CD47. In the hematopoietic system, SIRPα is mainly found on macrophages, dendritic cells, and granulocytes, while CD47 is present on most hematopoietic cells (Matozaki, T., Murata, Y., Okazawa, H. & Ohnishi, H. Functions and molecular mechanisms of the CD47-SIRPalpha signalling pathway. Trends Cell Biol. 19, 72-80 (2009)). It was shown that the murine Sirpa allele is highly polymorphic in the extracellular immunoglobulin V-like domain which interacts with CD47. Thirty-seven (37) unrelated normal human controls were sequenced and 4 polymorphisms were identified, suggesting that the Sirpa allele is polymorphic in humans as it is in mice (Takenaka et al. supra).

A large body of work has shown that human acute myeloid leukemia (AML) clones are hierarchically organized and maintained by leukemia initiating cells (AML-LSC) (Wang, J. C. & Dick, J. E. Cancer stem cells: lessons from leukemia. Trends Cell Biol. 15, 494-501 (2005)). However, little is known about molecular regulators that govern AML-LSC fate. CD47 is expressed in most human AML samples, but the level of expression on leukemic blasts varies. CD47 expression is higher on human AML LSCs compared to normal HSCs (Majeti, R. et al, CD47 is an adverse prognostic factor and therapeutic antibody target on human acute myeloid leukemia stem cells. Cell 138, 286 (2009)). Higher CD47 expression has been shown to be an independent poor prognostic factor in AML (Majeti et al., supra). Treatment of immune-deficient mice engrafted with human AML with a monoclonal antibody directed against CD47 results in reduction of leukemic engraftment in the murine bone marrow (Majeti et al., supra). However, it is not clear if this effect is specifically mediated through disruption of CD47-SIRPα interactions, as CD47 also binds to SIRPγ and to the integrin β3 subunit (Matozaki et al., supra).

### SUMMARY OF THE INVENTION

According to one aspect, there is provided a method for treating hematological cancer comprising modulating the interaction between human Sirpα and human CD47. Preferably, the interaction between human Sirpα and human CD47 is modulated by administering a therapeutically effective amount of a polypeptide capable of binding to the extracellular domain of human CD47.

According to a further aspect, there is provided a use of a compound for treating hematological cancer, the compound comprising a polypeptide capable of modulating the interaction between human Sirpα and human CD47 by binding to the extracellular domain of human CD47.

According to a further aspect, there is provided a use of a compound in the preparation of a medicament for treating hematological cancer, the compound comprising a polypeptide capable of modulating the interaction between human Sirpα and human CD47 by binding to the extracellular domain of human CD47.

In preferable aspects, the polypeptide comprises soluble human Sirpα, or a CD47 binding fragment thereof. In some embodiments, the polypeptide is the extracellular domain of human Sirpα.

In one embodiment the polypeptide is a Sirpα-Fc fusion protein, and is preferably SEQ ID NO. 13.

According to a further aspect, there is provided a method of determining genetic polymorphisms in humans affecting survival to hematological cancer, comprising:
a) sequencing the Sirpα gene from a plurality of humans having hematological cancer;
b) determining nucleotide differences in the Sirpα gene within the plurality of humans; and
c) correlating the nucleotide differences with survival to determine relevant polymorphisms.

In a further aspect, there is provided a method of prognosing likelihood of survival to hematological cancer comprising:
a) sequencing the Sirpα gene from the recipient; and
b) determining whether the relevant polymorphisms described herein exist.

According to some embodiments, the polypeptide capable of modulating the interaction between human Sirpα and human CD47 is selected from the group consisting of:
a) a polypeptide consisting of the amino acid sequence of SEQ ID NO. 1;
b) a polypeptide consisting of a CD47-binding fragment of the amino acid sequence of SEQ ID NO. 1, wherein the fragment comprises at least one of residues 31, 32, 34, 37, 74, 77, 83, 84, 86, 87, 90, 91, 96, 100, 102, 114, 118, 126 of SEQ ID NO. 1; and
c) a CD47-binding variant of one of the polypeptide in a) and b) with up to 1 amino acid insertion, deletion or substitution for every 7 amino acids in length of the polypeptide, wherein the polypeptide comprises at least one of residues 31, 32, 34, 37, 74, 77, 83, 84, 86, 87, 90, 91, 96, 100, 102, 114, 118, 126 of SEQ ID NO. 1.

According to another embodiment, the polypeptide capable of modulating the interaction between human Sirpα and human CD47 is selected from the group consisting of:
a) a polypeptide consisting of the amino acid sequence of SEQ ID NO. 2;
b) a polypeptide consisting of a CD47-binding fragment of the amino acid sequence of SEQ ID NO. 2; and
c) a CD47-binding variant of one of the polypeptide in a) and b) with up to 1 amino acid insertion, deletion or substitution for every 7 amino acids in length of the polypeptide;
wherein:
   i. at least one of residues at positions 31, 32, 34, 37, 74, 77, 83, 84, 86, 87, 90, 91, 96, 100, 102, 114, 118, 126 of SEQ ID NO. 2 in the polypeptide is replaced with corresponding residues 31, 32, 34, 37, 74, 77, 83, 84, 86, 87, 90, 91, 96, 100, 102, 114, 118, 126 of SEQ ID NO. 1; or
   ii. at least one of residues 129 and 130 of SEQ ID NO. 2 in the polypeptide is deleted.

According to another embodiment, the polypeptide capable modulating the interaction between human Sirpα and human CD47 is selected from the group consisting of:
a) a polypeptide consisting of an amino acid sequence selected from the group consisting of SEQ ID NOS. 4-7;
b) a polypeptide consisting of a CD47-binding fragment of an amino acid sequence selected from the group consisting of SEQ ID NOS. 4-7; and
c) a CD47-binding variant of one of the polypeptide in a) and b) with up to 1 amino acid insertion, deletion or substitution for every 7 amino acids in length of the polypeptide.

According to a further aspect, there is provided a pharmaceutical composition for treating a hematological cancer, preferably leukemia, and further preferably human acute myeloid leukemia (AML), comprising an effective amount of a polypeptide described herein and a pharmaceutically acceptable carrier.

In preferable aspects, the hematological cancer preferably comprises a CD47+ presenting cancer cell or tumour.

### BRIEF DESCRIPTION OF FIGURES

These and other features of the preferred embodiments of the invention will become more apparent in the following detailed description in which reference is made to the appended drawings wherein:

FIG. 1 shows NOD.SCID mice carrying the NOR-Idd13 locus (NOD.NOR-Idd13.SCID) do not support repopulation by human AML-LSC. Y axis displays percentage of human engraftment in the injected right femur (RF), marrow from bones at non-injected sites (BM), or spleen (Spl) of NOD.SCID (NS) and NOD.NOR-Idd13.SCID (Idd) mice 7-8 weeks after injection of primary AML cells from 4 patients. 3×10⁶ cells were introduced into each mouse either by intravenous (IV) or intrafemoral (IF) injection. Mice transplanted with the same patient sample are indicated by the same shape of symbol.

FIG. 2A shows defective homing of AML cells in NOD.NOR-Idd13.SCID mice. Percentage of human CD45+ engraftment in BM or spleen of sublethally irradiated NOD.SCID and NOD.NOR-Idd13.SCID (IDD) mice 16 hours after i.v. injection of primary AML cells, determined by flow cytometry. Each dot, square or triangle represents a single mouse. Bars indicate mean±SEM.

FIG. 2B shows AML-LSC function is held in check by innate immune cells. Primary cells from 10 AML patients with different leukemia subtypes and cytogenetic markers were transplanted i.f. into NOD.SCID or NOD.NOR-Idd13.SCID (IDD) mice pretreated with anti-murine CD122 antibody. The percentage of human engraftment in the injected right femur (RF), marrow from non-injected bones (BM) or spleen (SP) was determined 7-8 weeks later (Y axis). Compared to untreated mice (FIG. 1), repression of leukemic engraftment in IDD mice is reduced by pre-treatment with anti-CD122.

FIG. 3A and FIG. 3B show the effect of mouse innate immunity on engraftment of human acute myeloid leukemia (AML) in mouse xenograft. FIG. 3A: Modulation of natural killer (NK) cell and macrophage (MO) populations in NOD.SCID or NOD.NOR-Idd13.SCID. Surface phenotype of mouse NK cells and macrophages are indicated. FIG. 3B: Engraftment of human AML cells in NOD.SCID or NOD.NOR-Idd13.SCID (Idd) after macrophage depletion. Figures show percentage of human leukemic cells in harvested organs.

FIGS. 4A, 4B, 4C, and 4D show in vitro phagocytosis assay for human AML cells. FIG. 4A: CFSE-labeled human AML cells were co-incubated with NOD.SCID or NOD.NOR-Idd13.SCID mouse macrophages. After 2 hours macrophages were harvested and the percentage of F4/80+ mouse macrophages positive for CFSE was determined by flow cytometry. FIG. 4B: CFSE+/F4/80+ cells were sorted by fluorescent activated cell sorting and visualized using confocal microscopy. FIG. 4C: Co-cultures were pretreated with Cytochalasin D, which inhibits phagocytosis by inhibiting actin polymerization in macrophages. FIG. 4D: Expression of human CD45 in untreated or Cytochalasin D treated CFSE+/F4/80+ mouse macrophages, as indicated.

FIGS. 5A and 5B show that in vitro pre-incubation of human SIRPα (V2) fusion protein blocks homing of primary AML cells into NOD.SCID mouse bone marrow (BM) and spleen. FIG. 5A: The percentage of human CD44+ AML cells in BM and spleen was measured by flow cytometry using murine anti-human antibodies. Each symbol represents a different mouse. FIG. 5B: Homing efficiency of AML cells to NOD.SCID BM and spleen was calculated as [Total #AML cells recovered]/[Total #AML cells injected]×100.

FIG. 6 shows that in vitro treatment with human Sirpα fusion protein (hSirpα-Fc) decreases the repopulating ability of primary AML cells in NOD.SCID mice. Bars indicate the mean percentage of hCD45+ cells.

FIG. 7 shows that in vivo human Sirpα fusion protein (hSirpα-Fc) treatment decreases engraftment of primary AML cells in NOD.SCID mice. Stained cells were analyzed by flow cytometry to determine the engraftment levels in each mouse based on the percentage of hCD45+ cells. Bars indicate the average percentage of hCD45+ human cells in each group. Indicated P values are for hSirpα-Fc treatment compared to PBS-treatment.

FIGS. 8A and 8B illustrate the protein sequence alignment of murine SIRPα disclosed in WO 09/046541. cDNA prepared from BM-derived macrophages was used as a template for PCR amplification of Sirpα transcripts from NOD and NOR mice. The B6 mouse sequence is from the EnsEMBL database.

FIGS. 9A and 9B illustrate protein sequence alignments of murine and human SIRPα IgV domains disclosed in WO 09/046541. FIG. 9A: cDNA prepared from BM macrophages was used as a template for PCR amplification of Sirpα transcripts from NOD and NOR mice. The C57BL/6 (B6), BALB/c and 129/Sv sequences were obtained from EnsEMBL and NCBI databases. Open boxes represent b-pleated sheets identified in the X-ray crystal structure of SIRPα and correspond to similar regions in the Ig heavy chain variable region. Amino acids that vary between mouse strains are shaded. B6 was used as the parental sequence. FIG. 9B: Exon 3 of SIRPα containing the IgV domain was PCR amplified from genomic DNA of 37 individuals from the human HapMap phase 1 release. Open boxed regions represent the same features as in FIG. 9A, with V1 serving as the parental sequence.

### DETAILED DESCRIPTION

In the following description, numerous specific details are set forth to provide a thorough understanding of the invention. However, it is understood that the invention may be practiced without these specific details.

Applicant shows that CD47-SIRPα interaction modulates homing and engraftment of human AML-LSC in a xenotransplant model. Interruption of CD47-SIRPα signaling through targeting of either CD47 or SIRPα is a potential therapeutic approach for eradication of hematological CD47+ cancer cells and tumours, including cancer stem cells, such as AML-LSC in patients.

As used herein “cancer stem cell” refers to cancer cells found within tumors and hematological cancers, for example AML where the cancer stem cells are termed leukemic stem cells (AML-LSC), that are biologically distinct from the bulk tumor cells and possess characteristics associated with stem cells, specifically the ability to self renew and to propagate and give rise to all cell types found in a particular cancer sample.

As used herein “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

As used herein “engrafting” a cell, for example a cancer stem cell and preferably a human acute myeloid leukemic stem cell, means placing the stem cell into an animal, e.g., by injection, wherein the cell persists in vivo. This can be readily measured by the ability of the cancer stem cell, for example, to propagate.

As used herein “fragment” relating to a polypeptide or polynucleotide means a polypeptide or polynucleotide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule.

As used herein “fusion protein” refers to a composite polypeptide, i.e., a single contiguous amino acid sequence, made up of two (or more) distinct, heterologous polypeptides which are not normally or naturally fused together in a single amino acid sequence. Thus, a fusion protein may include a single amino acid sequence that contains two entirely distinct amino acid sequences or two similar or identical polypeptide sequences, provided that these sequences are not normally found together in the same configuration in a single amino acid sequence found in nature. Fusion proteins may generally be prepared using either recombinant nucleic acid methods, i.e., as a result of transcription and translation of a recombinant gene fusion product, which fusion comprises a segment encoding a polypeptide of the invention and a segment encoding a heterologous polypeptide, or by chemical synthesis methods well known in the art. Fusion proteins may also contain a linker polypeptide in between the constituent polypeptides of the fusion protein. The term “fusion construct” or “fusion protein construct” is generally meant to refer to a polynucleotide encoding a fusion protein. In one embodiment, the fusion protein is a polypeptide as described herein fused to a portion of an Ig molecule. The Ig portion of the fusion protein can include an immunoglobulin constant region, e.g. a human Cγ1 domain or a Cγ4 domain (e.g. the hinge, CH2, and CH3 regions of human IgCγ1 or human IgCγ4 (see e.g., Capon et al., U.S. Pat. Nos. 5,116,964; 5,580,756; 5,844,095, and the like). In one preferred embodiment, Ig fusion proteins include a polypeptide as described herein coupled to an immunoglobulin constant region (e.g., the Fc region).

In embodiments where the polypeptide is coupled to the Fc domain, the Fc domain may be selected from any immunoglobulin (e.g. an IgG such as IgG₁ or IgG2a or IgG4). Desirably, the selected Fc domain is modified (e.g. by amino acid substitution(s) at residues critical for binding with Fc receptors) to reduce or prevent binding to Fc receptors in vivo (i.e. the modified Fc domain preferably shows a reduced affinity for binding endogenous Fc receptors other than neonatal Fc receptors (FcRn), including, for example, FcγRI, FcγRII and FcγRIII). As well, the selected Fc domain is desirably modified to alter effector function, such as to reduce complement binding and/or to reduce or abolish complement dependent cytotoxicity. Such modifications have been extensively described by, for example, Clark and colleagues, who have designed and described a series of mutant IgG1, IgG2 and IgG4 Fc domains and their FcγR binding properties (Armour et al., 1999; Armour et al., 2002, the content of which is incorporated herein by reference in this application). For example, one or more amino acids at positions selected from 234, 235, 236, 237, 297, 318, 320 and 322 can be substituted to alter affinity for an effector ligand, such as an Fc receptor or the C1 component of complement, as reported in further detail by Winter et al in U.S. Pat. Nos. 5,624,821 and 5,648,260. Also, one or more amino acids at positions 329, 331 and 322 can be substituted to alter C1q binding and/or reduce or abolish CDC, as described for instance in U.S. Pat. No. 6,194,551. In one especially preferred modified Fc domain, the Fc domain is derived from IgG₁ (Wines et al., 2000) and comprises amino acid modification at amino acid 234 and/or 235, namely Leu²³⁴ and/or Leu²³⁵. These leucine residues are within the lower hinge region of IgG₁ where the Fc receptor engages with the Fc domain. One or both of the leucine residues may be substituted or deleted to prevent Fc receptor engagement (i.e. binding); for example, one or both of Leu²³⁴ and Leu²³⁵ may be substituted with alanine (i.e. L234A and/or L235A) or another suitable amino acid(s) (Wines et al., 2000).

In other embodiments, the half life of the fusion protein is improved by incorporating one more amino acid modifications, usually in the form of amino acid substitutions, for instance at residue 252, e.g., to introduce Thr, at residue 254, e.g., to introduce Ser, and/or at residue 256 e.g., to introduce Phe. Still other modifications can be made to improve half-life, such as by altering the CH1 or CL region to introduce a salvage receptor motif, such as that found in the two loops of a CH2 domain of an Fc region of an IgG. Such alterations are described for instance in U.S. Pat. Nos. 5,869,046 and 6,121,022.

Altered C1q binding, or reduced complement dependent cytotoxicity, can be introduced by altering constant region amino acids at locations 329, 331 and 322, as described in U.S. Pat. No. 6,194,551. The ability of the antibody to fix complement can further be altered by introducing substitutions at positions 231 and 239 of the constant region, as described in WO94/029351.

As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma.

The term “CD47+” is used with reference to the phenotype of cells targeted for binding by the present polypeptides. Cells that are CD47+ can be identified by flow cytometry using CD47 antibody as the affinity ligand. The cells examined for CD47 phenotype can include standard tumour biopsy samples including particularly blood samples taken from the subject suspected of harbouring CD47+ cancer cells.

The term “macrophage desupression” or “desupression of macrophages” as used herein refers to the desupression, removal of inhibition, increase or initiation of the role, activity and/or effect of macrophages.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “polypeptide” and “protein” are used interchangeably and mean proteins, protein fragments, modified proteins, amino acid sequences and synthetic amino acid sequences. The polypeptide can be glycosylated or not.

As used herein, “prognosing” as used herein means predicting or identifying the clinical outcome in a subject. A prognosis includes providing an indication of disease progression and also includes providing an indication of likelihood of death due to a disease or condition.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

According to one aspect, there is provided a method for treating hematological cancer comprising modulating the interaction between human Sirpα and human CD47. Preferably, the interaction between human Sirpα and human CD47 is modulated by administering a therapeutically effective amount of a polypeptide capable of binding to the extracellular domain of human CD47.

According to a further aspect, there is provided a use of a compound for treating hematological cancer, the compound comprising a polypeptide capable of modulating the interaction between human Sirpα and human CD47 by binding to the extracellular domain of human CD47.

According to a further aspect, there is provided a use of a compound in the preparation of a medicament for treating hematological cancer, the compound comprising a polypeptide capable of modulating the interaction between human Sirpα and human CD47 by binding to the extracellular domain of human CD47.

In some embodiments, the polypeptide comprises soluble human Sirpα, or a fragment thereof, preferably the polypeptide is the extracellular domain of human Sirpα.

In some embodiments, the polypeptide is fused to a second protein, preferably, the Fc portion of IgG. Preferably, the resulting fusion protein is SEQ ID NO. 13.

In some embodiments, the modulation results in desupression of macrophages.

In some embodiments, the hematological cancer is leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer is a lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma.

It is presently shown that interruption of SIRPα-CD47 interactions in AML results in impaired homing, engraftment, and migration of AML cells. These effects are mediated through improved innate immune surveillance by macrophages in the host bone marrow microenvironment. This therapeutic approach will likely be effective for other hematologic cancers that occupy a bone marrow microenvironmental niche.

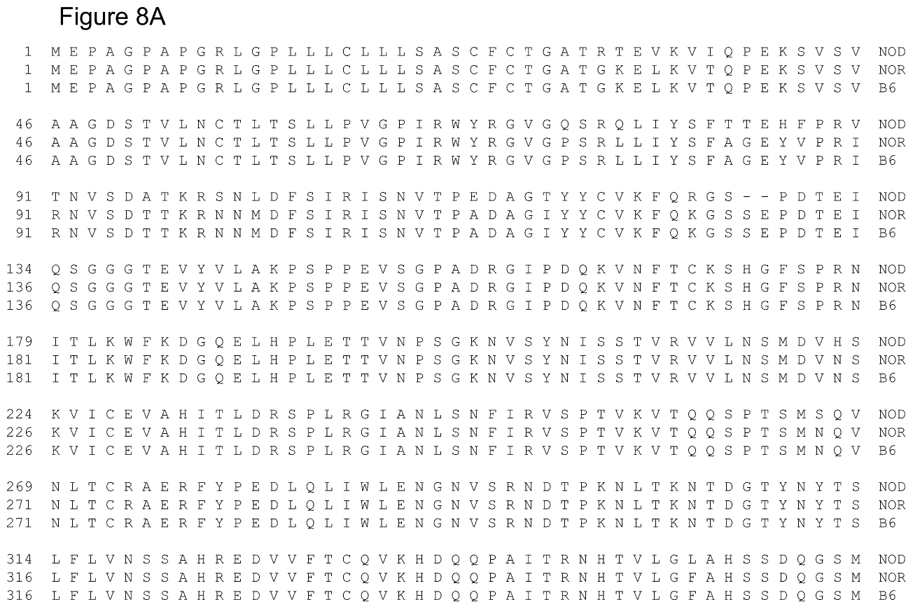

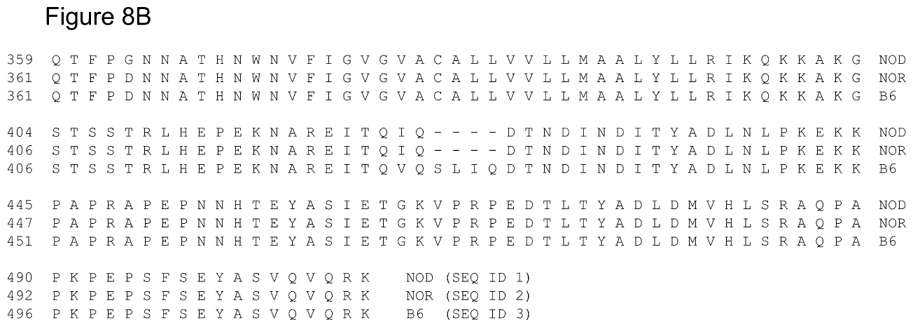

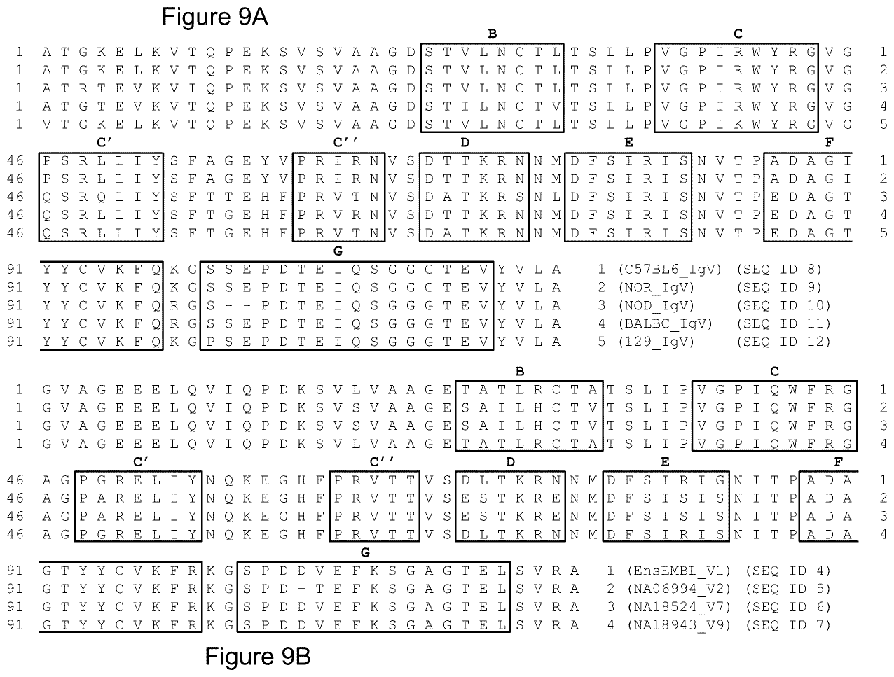

WO 09/065541 describes that polymorphisms in SIRPα confer differential capacity of NOD macrophages to support human hematopoiesis. The protein sequence alignments of SIRPα described in WO 09/065541 are presently reproduced as FIGS. 8A-8B and 9A-9B. cDNA prepared from BM-derived macrophages was used as a template for PCR amplification of Sirpα transcripts from NOD and NOR mice. Comparison of the Sirpα coding sequence between NOD and NOR revealed 24 amino acid differences, 20 of these in the extracellular IgV-like domain of molecule where the NOD sequence displays 18 substitutions and two deletions compared to NOR and B6. This observed variation in the Sirpα the N-terminal IgV-like domain between NOD and NOR or B6 is more extensive than that previously reported in this region amongst the B6, BALB/c and 129/Sv strains (Sano, S. et al. (1999) Biochem J 344 Pt 3, 667-75).

WO 09/065541 further describes that polymorphisms in SIRPα confers differential binding to human CD47. WO 09/065541 describes the sequencing of SIRPα IgV domain from 37 unrelated normal Caucasian (CEU), African (YRI), Chinese (CHB) and Japanese (JPT) individuals from the human HapMap genome project and identified 4 distinct SIRPα IgV alleles reflecting combinatorial variation at 18 amino acids, reproduced herein as FIGS. 9A and 9B. Applicants observed human allelic variations at predicted CD47 binding residues and in the same sub-regions of the SIRPα IgV domain that distinguish NOD from NOR alleles. WO 09/065541 further teaches that human CD47 binding to NOD-derived SIRPα IgV domain is very high and predictive of engraftment of human stem cells and that this effect is mediated through CD47-SIRPα signaling.

Accordingly there is provided, a method of determining genetic polymorphisms in humans affecting survival to hematological cancer, comprising:
a) sequencing the Sirpα gene from a plurality of humans having hematological cancer;
b) determining nucleotide differences in the Sirpα gene within the plurality of humans; and
c) correlating the nucleotide differences with survival to determine relevant polymorphisms.

In a further aspect, there is provided a method of prognosing likelihood of survival to hematological cancer comprising:
a) sequencing the Sirpα gene from the recipient; and
b) determining whether the relevant polymorphisms described herein exist.

In some embodiments, the nucleotide differences result in amino acid differences, preferably at least one of:
a) replacement of at least one of residues at positions 31, 32, 34, 37, 74, 77, 83, 84, 86, 87, 90, 91, 96, 100, 102, 114, 118, 126 of SEQ ID NO. 2 with corresponding residues 31, 32, 34, 37, 74, 77, 83, 84, 86, 87, 90, 91, 96, 100, 102, 114, 118, 126 of SEQ ID NO. 1; or b) deletion of at least one of residues 129 and 130 of SEQ ID NO. 2.

Further, according to some embodiments, the polypeptide capable modulating the interaction between human Sirpα and human CD47 is selected from the group consisting of:
a) a polypeptide consisting of the amino acid sequence of SEQ ID NO. 1;
b) a polypeptide consisting of a CD47-binding fragment of the amino acid sequence of SEQ ID NO. 1, wherein the fragment comprises at least one of residues 31, 32, 34, 37, 74, 77, 83, 84, 86, 87, 90, 91, 96, 100, 102, 114, 118, 126 of SEQ ID NO. 1; and
c) a CD47-binding variant of one of the polypeptide in a) and b) with up to 1 amino acid insertion, deletion or substitution for every 7 amino acids in length of the polypeptide, wherein the polypeptide comprises at least one of residues 31, 32, 34, 37, 74, 77, 83, 84, 86, 87, 90, 91, 96, 100, 102, 114, 118, 126 of SEQ ID NO. 1.

Preferably, the polypeptide is the CD47-binding fragment and comprises at least 3 consecutive amino acids in at least one of a region between residues 50-57, 63-71, 74-80, 88-92, 95-100, 103-109, 114-125 or 128-141, inclusive of SEQ ID NO. 1.

According to another embodiment, the polypeptide capable of modulating the interaction between human Sirpα and human CD47 is selected from the group consisting of:
a) a polypeptide consisting of the amino acid sequence of SEQ ID NO. 2;
b) a polypeptide consisting of a CD47-binding fragment of the amino acid sequence of SEQ ID NO. 2; and
c) a CD47-binding variant of one of the polypeptide in a) and b) with up to 1 amino acid insertion, deletion or substitution for every 7 amino acids in length of the polypeptide;
wherein:
   i. at least one of residues at positions 31, 32, 34, 37, 74, 77, 83, 84, 86, 87, 90, 91, 96, 100, 102, 114, 118, 126 of SEQ ID NO. 2 in the polypeptide is replaced with corresponding residues 31, 32, 34, 37, 74, 77, 83, 84, 86, 87, 90, 91, 96, 100, 102, 114, 118, 126 of SEQ ID NO. 1; or
   ii. at least one of residues 129 and 130 of SEQ ID NO. 2 in the polypeptide is deleted.

Preferably, the polypeptide is the CD47-binding fragment and comprises at least 3 consecutive amino acids in at least one of a region between residues 50-57, 63-71, 74-80, 88-92, 95-100, 103-109, 114-125 or 128-143, inclusive of SEQ ID NO. 2.

According to another embodiment, the polypeptide capable modulating the interaction between human Sirpα and human CD47 is selected from the group consisting of:
a) a polypeptide consisting of an amino acid sequence selected from the group consisting of SEQ ID NOS. 4-7;
b) a polypeptide consisting of a CD47-binding fragment of an amino acid sequence selected from the group consisting of SEQ ID NOS. 4-7; and
c) a CD47-binding variant of one of the polypeptide in a) and b) with up to 1 amino acid insertion, deletion or substitution for every 7 amino acids in length of the polypeptide.

Preferably, the polypeptide is the CD47-binding fragment and comprises at least 3 consecutive amino acids in at least one of a region between residues 24-31, 37-45, 48-54, 62-66, 69-74, 77-83, 88-99 or 102-116, inclusive, of any one of SEQ ID NOs. 4, 6 and 7; or between residues 24-31, 37-45, 48-54, 62-66, 69-74, 77-83, 88-99 or 102-115, inclusive, of SEQ ID NO. 5.

In some embodiments, the amino acid insertion, deletion or substitution is a conservative amino acid substitution. In other embodiments, there is no amino acid insertion, deletion or substitution.

In some embodiments, the polypeptide is the CD47-binding fragment and is between 6 and 30 amino acids in length, and in increasing preferability, between 8 and 28 amino acids in length, between 10 and 26 amino acids in length, between 12 and 24 amino acids in length, between 14 and 22 amino acids in length.

In some embodiments, the polypeptide is fused to a second polypeptide, preferably the Fc portion of IgG. In one embodiment the polypeptide is a Sirpα-Fc fusion protein, and is preferably SEQ ID NO. 13.

According to a further aspect, there is provided a pharmaceutical composition for treating a hematological cancer, preferably leukemia, and further preferably human acute myeloid leukemia (AML), comprising an effective amount of a polypeptide described herein and a pharmaceutically acceptable carrier.

The advantages of the present invention are further illustrated by the following examples. The examples and their particular details set forth herein are presented for illustration only and should not be construed as a limitation on the claims of the present invention.

### EXAMPLES

Mice

NOD/LtSz-Prdkc^{sc/sc} (NOD.SCID) (Shultz, L. D. et al. Multiple defects in innate and adaptive immunologic function in NOD/LtSz-scid mice. J Immunol 154, 180-191 (1995)) were bred and maintained under sterile conditions at the Ontario Cancer Institute (Toronto, Canada). NOD.NOR-Idd13 mice were maintained in either specific pathogen-free or barrier conditions at the Hospital for Sick Children (Toronto, Ontario). NOD.NOR-Idd13.SCID mice were generated from an intercross of NOD.NOR-Idd13 mice to NOD.SCID mice, followed by brother-sister matings screened by marker assisted genotyping until homozygosity was achieved at both Idd13 and SCID loci (Takenaka et al., supra).

Transplantation of Human Hematopoietic Cells into Mice

After informed consent was obtained, peripheral blood cells were obtained from patients with primary or secondary AML according to procedures approved by the Human Experimentation Committee. Low-density cells (less than 1.077 g/ml) were collected after separation on Ficoll-Hypaque (Pharmacia, Piscataway, NJ) and then cryopreserved in FCS containing 10% dimethyl sulfoxide.

8- to 10-week-old mice were sublethally irradiated with 275 cGy from a ¹³⁷Cs γ-irradiator 24 hours before transplantation of human cells. Mice were sacrificed 7-8 weeks post-transplantation, and murine BM and spleen was assessed for human cell engraftment by flow cytometric analysis for the presence of human CD45⁺ cells. In mice transplanted intrafemorally, the injected femur and the remaining bones (non-injected femur, tibias) were analyzed separately. In some experiments, mice were pretreated with 200 μg of anti-murine CD122 intraperitoneally after irradiation.

Flow Cytometry

A LSR II (BD) was used for flow cytometry. For analysis of human cell engraftment in mice, cells collected from mouse bone marrow were stained with phycoerythrin-Cy7-conjugated anti-human CD45 (HI.30; BD Pharmingen).

Human SIRP V1 and V2 Cloning into Type I TM Vector

Human SIRPα Variant 1 and 2 cDNA in pUC57 plasmid was PCR amplified in 5×HF Buffer using XhoI containing forward primer, BglII containing reverse primer and Phusion Hot start II High fidelity polymerase in order to obtain the complete IgV domain of SIRPα variant 1 and 2.

pUC57 SIRPα vector were subjected to restriction digest with XhoI and BglII. pINFUSE-hIgG4-Fc1 was digested with EcoRI and BglII and the SIRPα insert was ligated into pINFUSE-hIgG4-Fc1 using LigaFast Rapid DNA ligation System from Promega™.

The resulting pINFUSE-hIgG4-Fc1-human SIRPα vector was transformed into One Shot TOP10 competent E. coli from Invitrogen.

Transfection

Plasmid DNA and fectin were diluted in an appropriate volume of OptiMeM and mixed. FreeStyle™ 293-F cells were transfected at 37° C. incubator with a humidified atmosphere of 8% CO² in air. Cells or media was were harvested 4 to 6 days after transfection.

Protein Harvest

Fc protein was harvested from 293F culture. The culture was spun and the supernatant collected and filtered through a PES 0.45 um filter, then through a PES 0.22 um filter. The supernatant was concentrated using Centricon-70 mL centrifugation filters at ˜3500×g for 10-20 mins and eluted in a G column at 4° C. The protein was collected in 1M Tris HCl pH 8.0. The sample was further desalted by centrifugation and resuspended in PBS.

### Example 1

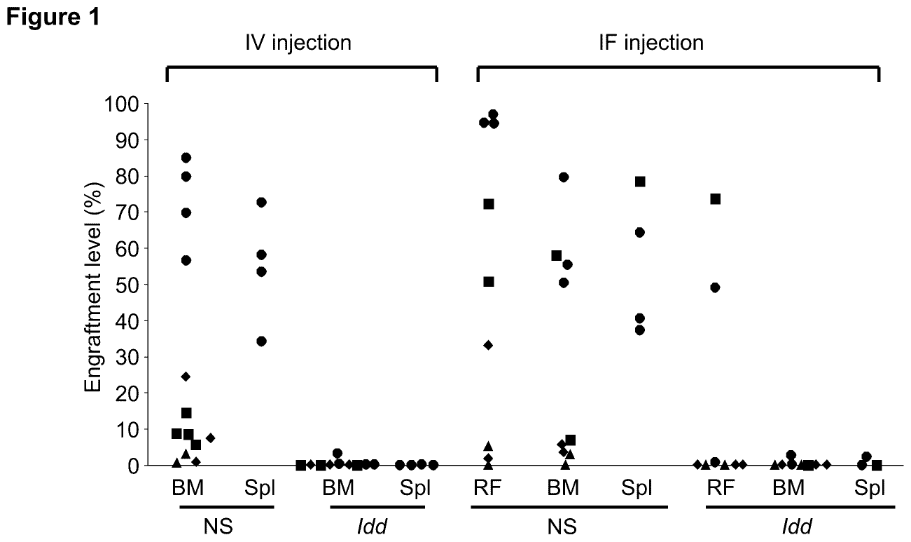

To investigate the relevance of CD47-SIRPα interaction in primary human AMLs, we transplanted primary cells from three AML patients intravenously (i.v.) into NOD.SCID and NOD.NOR-Idd13.SCID (Idd) mice. None of the AML samples could engraft the Idd mice while robust engraftment in the NOD.SCID mice was observed (FIG. 1), confirming the data obtained previously with normal human HSCs. When the same samples were transplanted intrafemorally (i.f.), 2 of 6 Idd mice showed engraftment in the injected femur but no engraftment in other bones or the spleen (FIG. 1).

### Example 2

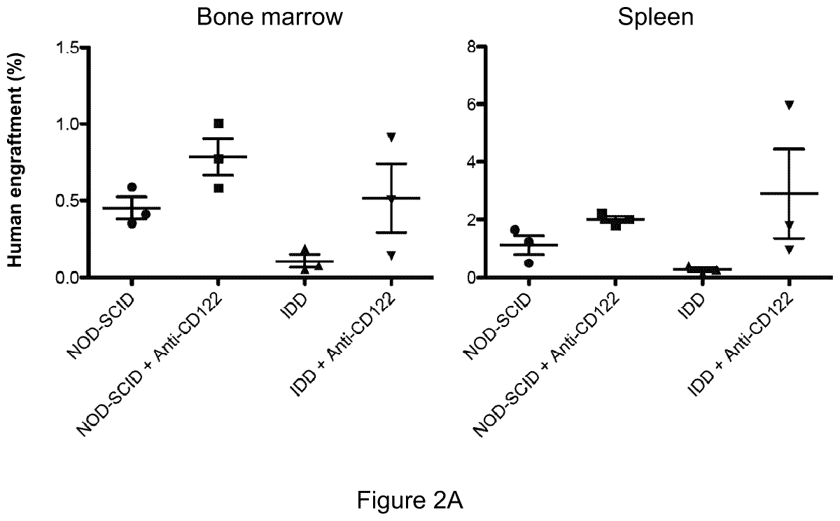

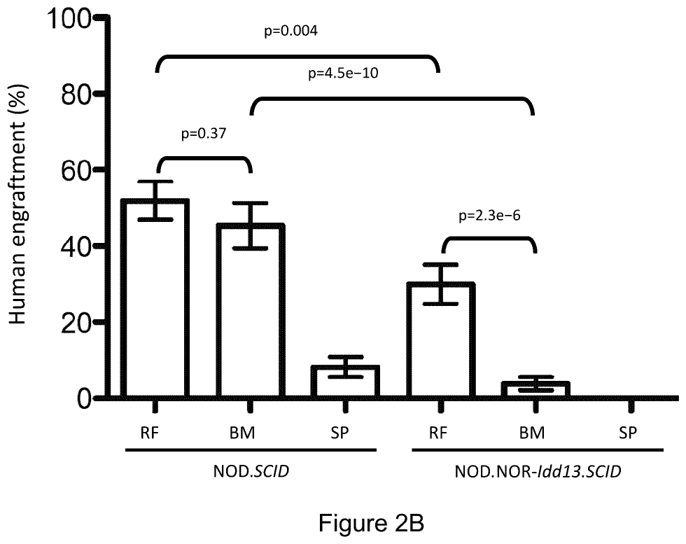

We next performed i.f. transplants into mice pre-treated with antibody directed against murine CD122 which depletes host natural killer (NK) cells and some macrophages. Engraftment in the injected femur was observed for all 10 AML samples tested in NOD.SCID mice (43/43, 100%), and interestingly in 31 of 42 (74%) Idd mice (8/10 AML samples tested) (FIG. 2B), however the engraftment level was lower in Idd mice compared to NOD.SCID mice. In contrast to results obtained without anti-CD122 pre-treatment, engraftment in non-injected bones was detectable in 8/42 Idd mice (19%, 2/10 AML samples tested) while as expected most of the transplanted NOD.SCID mice supported migration (38/43, 88%, 10/10 AML samples tested). However the engraftment level in non-injected bones was significantly lower in Idd compared to NOD.SCID mice. Moreover, AML-LSCs were unable to repopulate the spleens of Idd mice. Homing assays performed with the best engrafting AML sample in this study revealed severe homing deficiencies in Idd compared to NOD.SCID mice (FIG. 2A).

### Example 3

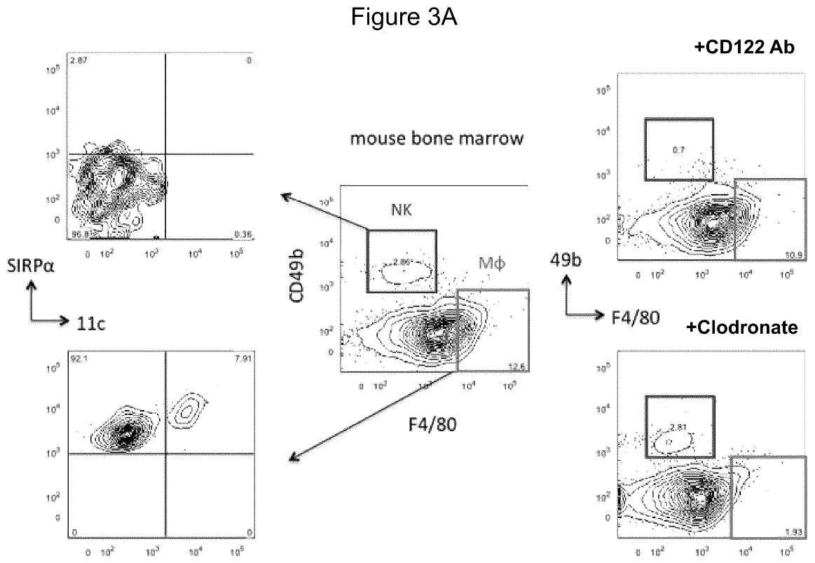

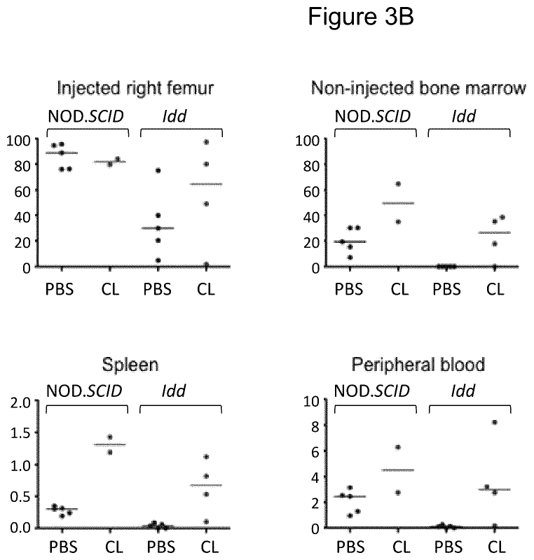

We studied the effect of mouse innate immunity on engraftment of human acute myeloid leukemia (AML) in mouse xenograft. FIG. 3A shows the modulation of natural killer (NK) cell and macrophage (MO) populations in NOD.SCID or NOD.NOR-Idd13.SCID. Surface phenotype of mouse NK cells and macrophages are indicated. Macrophages but not NK cells express Sirpα (left panels). Mice were treated with intraperitoneal (i.p.) injections of antibody against CD122 (expressed mainly on NK cells) or Clodronate. CD122 antibody treatment, which has been shown to improve engraftment of human hematopoietic cells in NOD.SCID mice, leads to rapid NK cell depletion but does not reduce mouse macrophages (upper right panel). Treatment with the bisphosphonate Clodronate leads to apoptosis and efficient depletion of macrophages (lower right panel of FIG. 3A).

FIG. 3B shows the engraftment of human AML cells in NOD.SCID or NOD.NOR-Idd13.SCID (Idd) after macrophage depletion. Sublethally irradiated mice were treated with PBS (controls) or Clodronate (CL) i.p. 48 hours prior to intrafemoral transplantation of human AML cells. Clodronate treatment was continued on a weekly basis until mice were sacrificed 8 weeks after transplantation. Injected bone marrow, non-injected bone marrow, spleen and peripheral blood of mice were harvested and human leukemic engraftment was assessed by flow cytometry using human specific markers. Figures show percentage of human leukemic cells in harvested organs. There is reduced engraftment in the injected femur of PBS-treated control Idd mice compared to NOD.SCID mice and no engraftment at other sites; engraftment at all sites in Idd mice was observed after Clodronate treatment. These findings support the hypothesis that SIRPα-CD47 interactions are critical for AML engraftment, and that decreased engraftment of AML cells in Idd mice is mediated by macrophages.

### Example 4

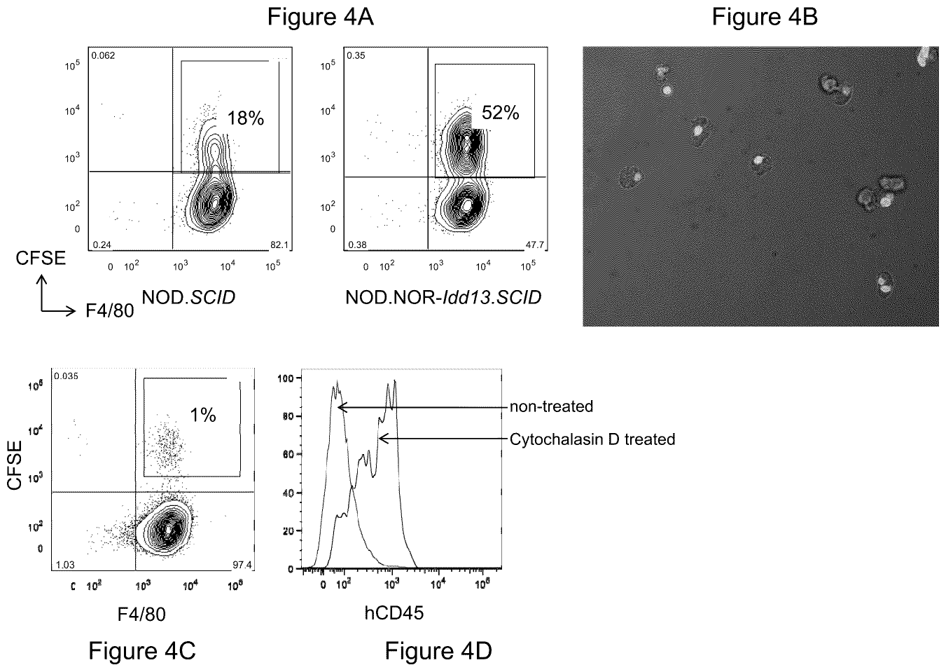

In vitro phagocytosis assay for human AML cells was performed. CFSE-labeled human AML cells were co-incubated with NOD.SCID or NOD.NOR-Idd13.SCID mouse macrophages. After 2 hours macrophages were harvested and the percentage of F4/80+ mouse macrophages positive for CFSE was determined by flow cytometry. CFSE-positivity of mouse macrophages suggests engulfment of human CFSE+ AML cells (FIG. 4A). NOD.NOR-Idd13.SCID-AML co-cultures consistently showed higher percentages of CFSE+/F4/80+ compared to NOD.SCID-AML co-cultures. This suggests that the lack of interaction between CD47 on AML cells and Sirpα on mouse macrophages results in increased phagocytosis of AML cells.

Following fluorescent activated cell sorting, CFSE+/F4/80+ cells were visualized using confocal microscopy. FIG. 4B shows one informative field with CFSE+ cells engulfed by macrophages.

Co-cultures were pretreated with Cytochalasin D, which inhibits phagocytosis by inhibiting actin polymerization in macrophages. Cytochalasin D treatment significantly reduced the percentage of CFSE+/F4/80+ cells (FIG. 4C).

FIG. 4D shows the expression of human CD45 in untreated or Cytochalasin D treated CFSE+/F4/80+ mouse macrophages, as indicated. Low expression of human CD45 suggests engulfment of human AML cells in mouse macrophages as human antibodies are unable to bind to engulfed cells. These results confirm that the majority of untreated CFSE+/F4/80+ cells are macrophages that have ingested human AML cells (CD45 negative). Overall, these findings suggest that SIRPα-CD47 interactions are critical for AML cells to evade innate immune attack by macrophages.

### Example 5

Example 5 demonstrates that in vitro pre-incubation of human SIRPα (V2) fusion protein blocks homing of primary AML cells into NOD.SCID mouse bone marrow (BM) and spleen. Primary cells harvested from AML Pt9601 were incubated with or without human SIRPα-Fc fusion protein at a concentration of 50 μg/ml in IMDM+15% BIT for 2 hours at 37° C. Cells incubated with (hSIRPα-Fc) or without (No treatment) fusion protein were harvested and transplanted intravenously into sublethally irradiated NOD.SCID mice. Sixteen hours post transplantation, mice were sacrificed and cells were harvested from BM and spleen.

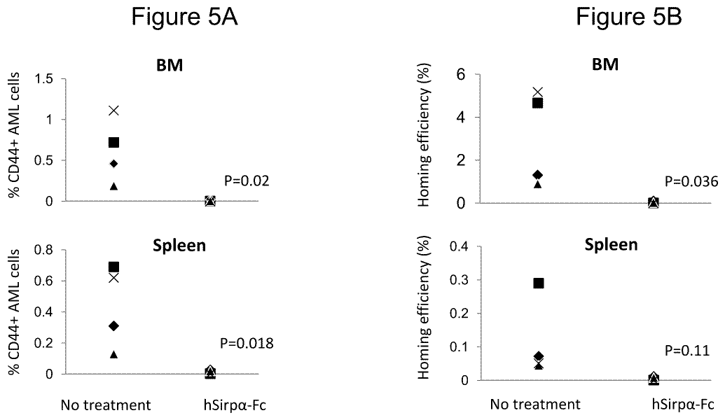

The percentage of human CD44+ AML cells in BM and spleen was measured by flow cytometry using murine anti-human antibodies. In vitro incubation with human SIRPα-Fc significantly decreased the percentage of human CD44+ AML cells in both BM (P=0.02) and spleen (P=0.018), compared to the untreated group (FIG. 5A—each symbol represents a different mouse).

Homing efficiency of AML cells to NOD.SCID BM and spleen was calculated as [Total #AML cells recovered]/[Total #AML cells injected]×100. Human SIRPα-Fc treatment significantly reduced the homing efficiency of AML cells to NOD.SCID BM (P=0.036) and also to spleen (NS) (FIG. 5B).

### Example 6

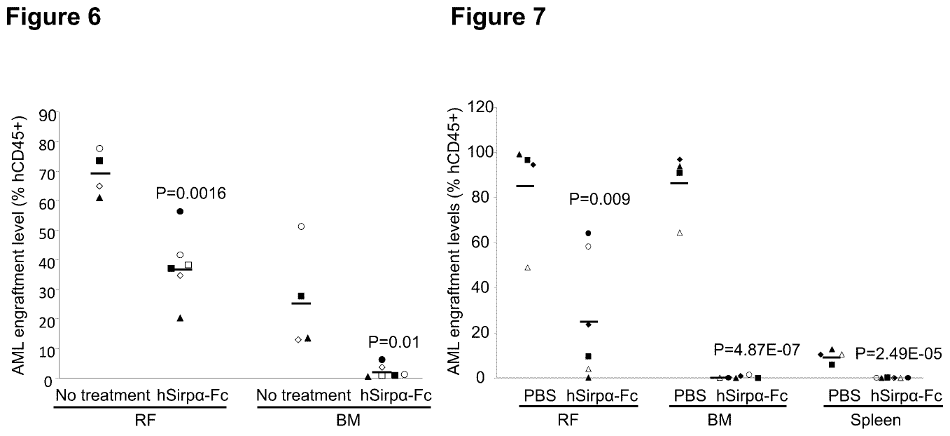

Example 6 shows that in vitro treatment with human Sirpα fusion protein (hSirpα-Fc) decreases the repopulating ability of primary AML cells in NOD.SCID mice. Primary AML cells from Pt90181 (unclassified AML) were incubated with or without hSirpα-Fc at a concentration of 50 μg/ml in IMDM+15% BIT for 2 hours at 37° C. After incubation, cells were harvested and transplanted intrafemorally into sublethally irradiated NOD.SCID mice at 2.7×10⁶ cells per mouse. 4 weeks post transplantation, mice were sacrificed and cells were harvested from the injected femur (RF) and uninjected femur (BM) for staining with anti-human antibodies. Stained cells were analyzed by flow cytometry to determine the engraftment levels in each individual mouse based on the percentage of hCD45+ cells (FIG. 6). Bars indicate the mean percentage of hCD45+ cells. Pre-incubation of AML cells with hSirpα-Fc significantly decreased AML engraftment levels in both RF (P=0.0016) and BM (P=0.01) compared to untreated controls.

### Example 7

Example 7 demonstrates that in vivo human Sirpα fusion protein (hSirpα-Fc) treatment decreases engraftment of primary AML cells in NOD.SCID mice. Primary AML cells from patient 0285 (FAB M2) were injected intrafemorally into sublethally irradiated NOD.SCID mice. Starting 10 days post transplantation, hSirpα-Fc was administered intraperitoneally at a dose of 200 μg per mouse (8 mg/kg), 3 times/week for 7 doses. Mice were then sacrificed and cells harvested from the injected femur (RF), uninjected femur (BM) and spleen for staining with anti-human antibodies. Stained cells were analyzed by flow cytometry to determine the engraftment levels in each mouse based on the percentage of hCD45+ cells (FIG. 7). Bars indicate the average percentage of hCD45+ human cells in each group. Indicated P values are for hSirpα-Fc treatment compared to PBS-treatment. Treatment with hSirpα-Fc dramatically decreased AML engraftment levels in all tissues analyzed. Profound reduction of AML cells in BM and spleen to almost undetectable levels suggests that hSirpα-Fc completely inhibited AML stem cell migration from the injected femur to other hematopoietic sites.

### DISCUSSION

Here we have shown that human AML-LSC have significantly reduced engraftment ability in NOD.NOR-Idd13.SCID mice, in concordance with the data obtained with normal hematopoietic cells. Our results are consistent with those obtained with anti-CD47 treatment of mice engrafted with AML (Majeti et al., supra), and support the hypothesis that attenuation of CD47-SIRPα interaction (as in Idd mice) impairs AML-LSC function. This effect is somewhat ameliorated by depletion of NK cells and macrophages through anti-CD122 treatment, enabling engraftment in the injected femur and in some cases migration to other bones. This suggests that at least some of the anti-leukemic activity in vivo may be mediated by cells of the innate immune system, in particular macrophages expressing SIRPα. Our findings further suggest that SIRPα-CD47 interactions are critical for AML cells to evade innate immune attack by macrophages. Interruption of CD47-SIRPα signaling through targeting of either CD47 or SIRPα is a therapeutic approach for eradication of hematological cancers cells such as leukemic stem cells, preferably AML-LSC. We demonstrate that human SIRPα (V2) fusion protein blocks homing of primary AML cells into NOD.SCID mouse bone marrow (BM) and spleen and decreases the repopulating ability of primary AML cells in NOD.SCID mice. Notably, we demonstrate that in vivo human Sirpα fusion protein (hSirpα-Fc) treatment decreases engraftment of primary AML cells in NOD.SCID mice.

Although preferred embodiments of the invention have been described herein, it will be understood by those skilled in the art that variations may be made thereto without departing from the spirit of the invention or the scope of the appended claims. All documents disclosed herein are incorporated by reference.

1. A method of determining genetic polymorphisms in humans affecting survival to a hematological cancer, comprising:
a) sequencing the Sirpα gene from a plurality of humans having the hematological cancer;
b) determining nucleotide differences in the Sirpα gene within the plurality of humans; and
c) correlating the nucleotide differences with survival to determine relevant polymorphisms.

2. The method of claim 1, wherein the nucleotide differences result in amino acid differences.

3. A method of prognosing likelihood of survival of a patient with a hematological cancer comprising:
a) sequencing the Sirpα gene from the patient; and
b) determining whether the relevant a polymorphism that results in an amino acid difference in the Sirpα gene from the patient, wherein the amino acid difference is at least one of:
(i) replacement of at least one of residues at positions 31, 32, 34, 37, 74, 77, 83, 84, 86, 87, 90, 91, 96, 100, 102, 114, 118, and 126 of SEQ ID NO: 2 with corresponding residues 31, 32, 34, 37, 74, 77, 83, 84, 86, 87, 90, 91, 96, 100, 102, 114, 118, and 126 of SEQ ID NO: 1; or
(ii) deletion of at least one of residues 129 and 130 of SEQ ID NO: 2.


SEQUENCE LISTING (13 sequences)

SEQ ID NO: 1 - protein, 507 aa, Mus musculus
MEPAGPAPGRLGPLLLCLLLSASCFCTGATRTEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVLAKPSPPEVSGPADRGIPDQKVNFTCKSHGFSPRNITLKWFKDGQELHPLETTVNPSGKNVSYNISSTVRVVLNSMDVHSKVICEVAHITLDRSPLRGIANLSNFIRVSPTVKVTQQSPTSMSQVNLTCRAERFYPEDLQLIWLENGNVSRNDTPKNLTKNTDGTYNYTSLFLVNSSAHREDVVFTCQVKHDQQPAITRNHTVLGLAHSSDQGSMQTFPGNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 2 - protein, 509 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 3 - protein, 513 aa, Mus musculus
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 4 - protein, 120 aa, Homo sapiens
GVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 5 - protein, 119 aa, Homo sapiens
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRA

SEQ ID NO: 6 - protein, 120 aa, Homo sapiens
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDDVEFKSGAGTELSVRA

SEQ ID NO: 7 - protein, 120 aa, Homo sapiens
GVAGEEELQVIQPDKSVLVAAGETATLRCTATSLIPVGPIQWFRGAGPGRELIYNQKEGHFPRVTTVSDLTKRNNMDFSIRISNITPADAGTYYCVKFRKGSPDDVEFKSGAGTELSVRA

SEQ ID NO: 8 - protein, 119 aa, Mus musculus
ATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLA

SEQ ID NO: 9 - protein, 119 aa, Mus musculus; identical to SEQ ID NO: 8

SEQ ID NO: 10 - protein, 117 aa, Mus musculus
ATRTEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVLA

SEQ ID NO: 11 - protein, 119 aa, Mus musculus
ATGTEVKVTQPEKSVSVAAGDSTILNCTVTSLLPVGPIRWYRGVGQSRLLIYSFTGEHFPRVRNVSDTTKRNNMDFSIRISNVTPEDAGTYYCVKFQRGSSEPDTEIQSGGGTEVYVLA

SEQ ID NO: 12 - protein, 119 aa, Mus musculus
VTGKELKVTQPEKSVSVAAGDSTVLNCTLTSLLPVGPIKWYRGVGQSRLLIYSFTGEHFPRVTNVSDATKRNNMDFSIRISNVTPEDAGTYYCVKFQKGPSEPDTEIQSGGGTEVYVLA

SEQ ID NO: 13 - protein, 368 aa, Mus musculus
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK